(19) United States
(12) Patent Application Publication
(10) Pub. No.: US 2008/0033446 A1
(43) Pub. Date: Feb. 7, 2008

(54) Flexible and breathable filler for medical application

(76) Inventor: Kwan-Ku Lin, Taipei (TW)

Correspondence Address: BACON & THOMAS, PLLC, 625 SLATERS LANE, FOURTH FLOOR, ALEXANDRIA, VA, 22314, US

(21) Appl. No.: 11/892,048

(22) Filed: Aug. 20, 2007

Related U.S. Application Data
(63) Continuation-in-part of application No. 10/384,533, filed on Mar. 11, 2003, now abandoned.

(51) Int. Cl. A61F 2/28

(52) U.S. Cl. 606/92

(57) ABSTRACT

A medical filler comprises a flexible and breathable filling member, and a pasty medicinal material contained in the filling member. The filling member is formed of one or more meshed walls. The filling member is compressed prior to injection of the pasty medicinal material into the filling member. The injecting of the pasty medicinal material causes the filling member to regain its original form after the filling member being inserted into a cancellous portion of a bone receiving treatment.

### CROSS-REFERENCE TO RELATED APPLICATION

The present application is a continuation-in-part application of U.S. patent application Ser. No. 10/384,533, filed Mar. 11, 2003. The entire contents of which are incorporated herein by reference.

### FIELD OF THE INVENTION

The present invention relates generally to a medicinal filler, and more particularly to a method for implanting a medicinal filler into a bone.

### BACKGROUND OF THE INVENTION

The conventional medical blockage of cavity often involves the use of a solid material, such as gypsum. Similarly, the fixation of an incision involves the use of a solid material. Such solid fillers as described above are used to bring about a fixation effect. However, the conventional medical fillers often fail to join together intimately, thereby resulting in formation of gaps among them. In another words, they are bound to loosen up easily due to poor frictional contact. The conventional cavity blockage is defective in design in that the filler and the cavity wall lack an intimate contact. In the case of the incision fixation, the filler is not properly shaped to fit well into the incision.

U.S. Pat. No. 5,549,679 discloses a method for stabilizing a spinal motion segment by boring and excising an opening in a disc nucleus, inserting a fabric bag having a pore size between about 0.25 and 5.00 mm, packing the bag with a graft medium until the bag is a rigid self-retaining shape, then closing the bag. The nucleus is excised by inserting an expandable cutting tool in order to cut an area greater than the access opening. A further spinal stabilization includes positioning a flexible bag containing graft medium against the exterior of the lamina bone of the adjacent vertebra.

US patent publication No. 2002/0068974 A discloses expandable fabric bags and methods of use for reduction, fixation, support and treatment of bone abnormalities such as tibial plateau fractures, femoral head necrosis, compression fractures of the spine and other bone abnormalities. A cavity forming tool is used to form a cavity in a bone for accommodating the expandable fabric bag.

In the prior art the expandable bags have a pore size of about 0.25 mm or greater, and a sever leakage problem will occur when inflation of the expandable bags is carried out by injecting a pasty material formed of a mixture of liquid and a powdery bone cement, so that it is essential to create and expand a cavity in a cancellous portion of a bone receiving treatment when an access opening is formed on the bone, thereby the expandable bags can be inserted into and inflated in the expanded cavity.

### SUMMARY OF THE INVENTION

The primary objective of the present invention is to provide a method for implanting a medicine filler in a bone, which is free of the drawbacks of the conventional implantation methods. The filler comprises a pasty medicinal material and a filling member which is flexible and breathable. The filling member is properly compressed before it is injected with the pasty medicinal material. Upon having been injected, the filling member returns to its original form.

The present invention provides a method for implanting a medical filler into a bone, which comprises the following steps:

providing a flexible and breathable filling member formed of one or more meshed walls and provided with a hollow receiving portion and an inlet in communication with said receiving portion, said meshed walls being elastically compressible and a single-layer wall or a laminated multi-layer wall, and each layer of which is provided with more than 100 meshes per linear inch, so that said meshed walls are not air-tight;

forming an access opening on a bone so that a cancellous portion thereof is exposed;

directly inserting the filling member into the access opining so that the filling member is in the cancellous portion of the bone without using a tool to create a cavity larger than the access opening in the cancellous portion of the bone; and

injecting a pasty medicinal material into said receiving portion via said inlet of said filling member.

Preferably, said meshed walls are of a single-layer wall construction.

Preferably, said meshed walls are of a laminated multi-layer wall construction, and the meshes of each layer of the multi-layer walls are not aligned.

Preferably, said filling member is integrally made of one meshed wall such that said filling member takes the form of a sac, bag, or ball.

Preferably, said pasty medicinal material is a mixture of a liquid and a bone cement. More preferably, said bone cement is selected from the group consisting of gypsum, calcium sulfate, calcium phosphate, and hydroxy apatite.

Preferably, the method of the present invention further comprises fastening detachably an injection tool with said inlet of said filling member, so that said pasty medicinal material is injected into said receiving portion via said inlet of said filling member by said injection tool. More preferably, said injecting tool comprises a connection tube and an injecting cylinder, said connection tube being provided at one end with an outer threaded portion such that said outer threaded portion of said connection tube is engaged with an inner threaded portion of said inlet of said filling member; said injecting cylinder comprising a barrel and a plunger, said barrel being fastened with another end of said connection tube, wherein said barrel is used to hold said pasty medicinal material which is to be injected into said receiving portion of said filling member via said connection tube by a plunging motion of said plunger.

### BRIEF DESCRIPTION OF THE DRAWINGS

FIG. 1 shows a perspective view of a medicine filling system of the present invention.

FIG. 2 shows an exploded view of the medicine filling system of the present invention.

FIG. 3a shows a sectional schematic view of a meshed wall of the filling member of the present invention.

FIG. 3b shows a sectional schematic view of a laminated wall of the filling member of the present invention.

FIGS. 4a-4d are schematic view of the filling members of the present invention in various forms.

FIGS. 5a-5C are schematic views of the system of the present invention at work to bring about a blocking action in the surgical operation.

### DETAILED DESCRIPTION OF THE INVENTION

The present invention is characterized by the medicinal material which is injected in a pasty form, and by the filling member which is formed of one or more flexible and breathable walls. These walls are provided with a plurality of passages and are single-layer walls or multi-layer walls made by laminating. The filling member is provided with a hollow receiving portion and an inlet via which the pasty medicinal material is injected into the receiving portion. Prior to injection of the pasty medicinal material into the filling member, the filling member is compressed. The injection action causes the filling member to return to its original form. The interior of the receiving portion of the filling member is in communication with the exterior of the filling member via the passages. As far as the passages are concerned, the filling member of the present invention is formed of the single-layer walls or the multi-layer walls, each layer having meshes in the range of 20-100 per linear inch. If the filling member is formed of a 20-mesh wall, three or more layers laminated walls are suggested, depending on the viscosity of the pasty medicinal material and the content of grains in the medicinal material. For example, if the pasty medicinal material is poly(methyl methacrylate) (PMMA), the filling member is preferably formed of three layers of 20-mesh walls by laminating. This is due to the fact that PMMA has a relatively high viscosity and contains grains which are relatively large in size. If the pasty medicinal material has a relatively low viscosity and contains grains which are relatively small in size, the filling member is preferably formed of four or more layers of 20-mesh walls by laminating. The laminated structure of the filling member of the present invention is dependent on the viscosity of the pasty medicinal material or the particle size of grains contained therein, as exemplified by the experimental data listed in the following table.

| | | No. of | Form of | | |
|---|---|---|---|---|---|
| | Type of | layers of | filling | Medicinal | Leaking |
| Experiment | wall | walls | member | material | or not |
| 1 | 20-mesh | 3 | baglike, | PMMA | No leak |
| | wall | | meshed | | |
| 2 | 20-mesh | 3 | baglike, | gypsum | slight |
| | wall | | meshed | paste | leak |
| 3 | 20-mesh | 4 | baglike, | gypsum | No leak |
| | wall | | meshed | paste | |
| 4 | 50-mesh | 2 | baglike, | PMMA | No leak |
| | wall | | meshed | | |
| 5 | 50-mesh | 3 | baglike, | PMMA | No leak |
| | wall | | meshed | | |
| 6 | 100-mesh | 1 | baglike, | PMMA | No leak |
| | wall | | non-woven | | |
| | | | fabric | | |

The filling member of the present invention is filled with the pasty medicinal material by means of an injecting tool, such as an electric or manual injection cylinder. The pasty medicinal material is preferably capable of hardening. The recommended medicinal materials include bone cement, PMMA, and bone substitute such as gypsum and calcium phosphate.

The flexible and breathable filler of the present invention is used mainly for the medical blockage, the medical fixation, or the press of the soft tissues. The pasty medicinal material of the filler may be impregnated with a medical substance for treating disease or relieving pain. The medical substance is slowly released by the medicinal material.

The filling member of the present invention may be integrally formed of the single-layer walls or the multi-layer walls, or may be formed by connecting pieces of the single-layer walls or the multi-layer walls. If the filling member is made with the multi-layer walls, the meshes of each layer of the multi-layer walls are not necessarily aligned. The filling member of the present invention is made of a biocompatible material, such as titanium, or a biodegradable material.

The features and the advantages of the present invention will be more readily understood upon a thoughtful deliberation of the following detailed description of the preferred embodiments of the present invention with reference to the accompanying drawings.

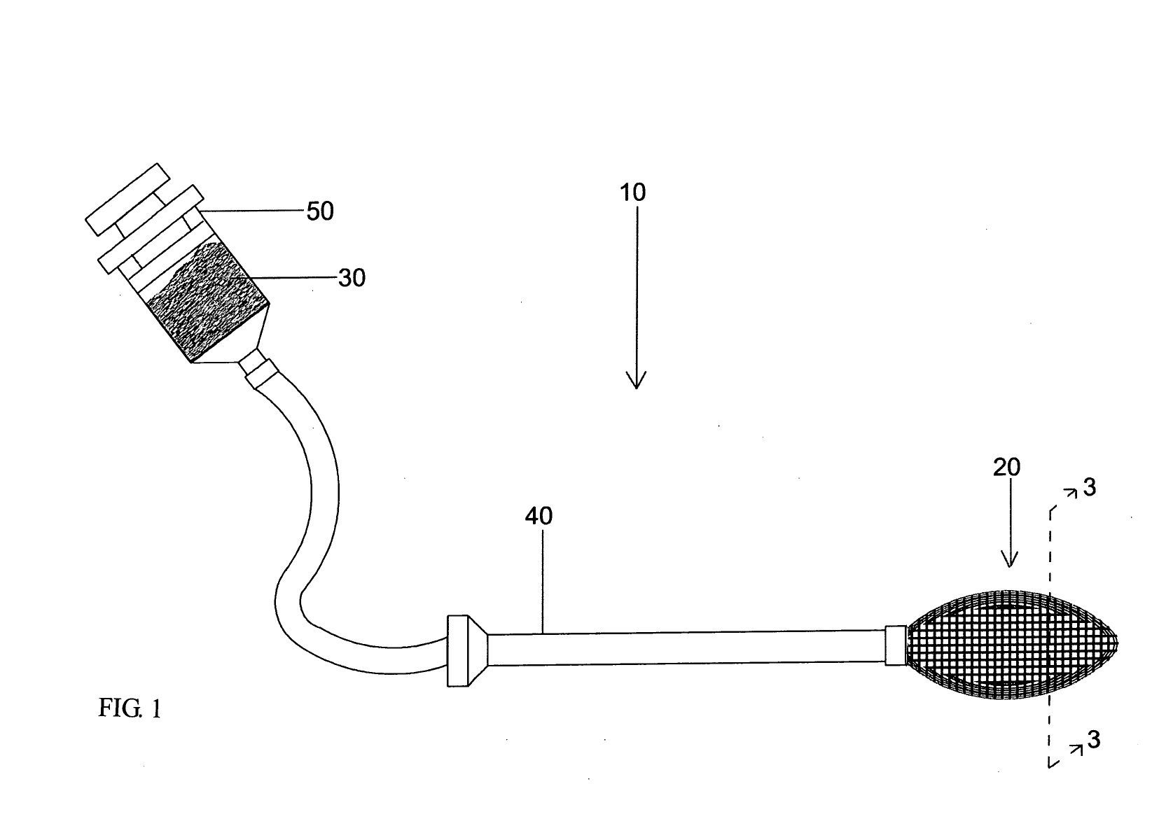

As shown in FIG. 1, a medical filler system 10 embodied in the present invention comprises a flexible and breathable filling member 20, a pasty medicinal material 30, a connection tube 40, and an injecting cylinder 50 by which the pasty medicinal material 30 is injected into the filling member 20 via the connection tube 40.

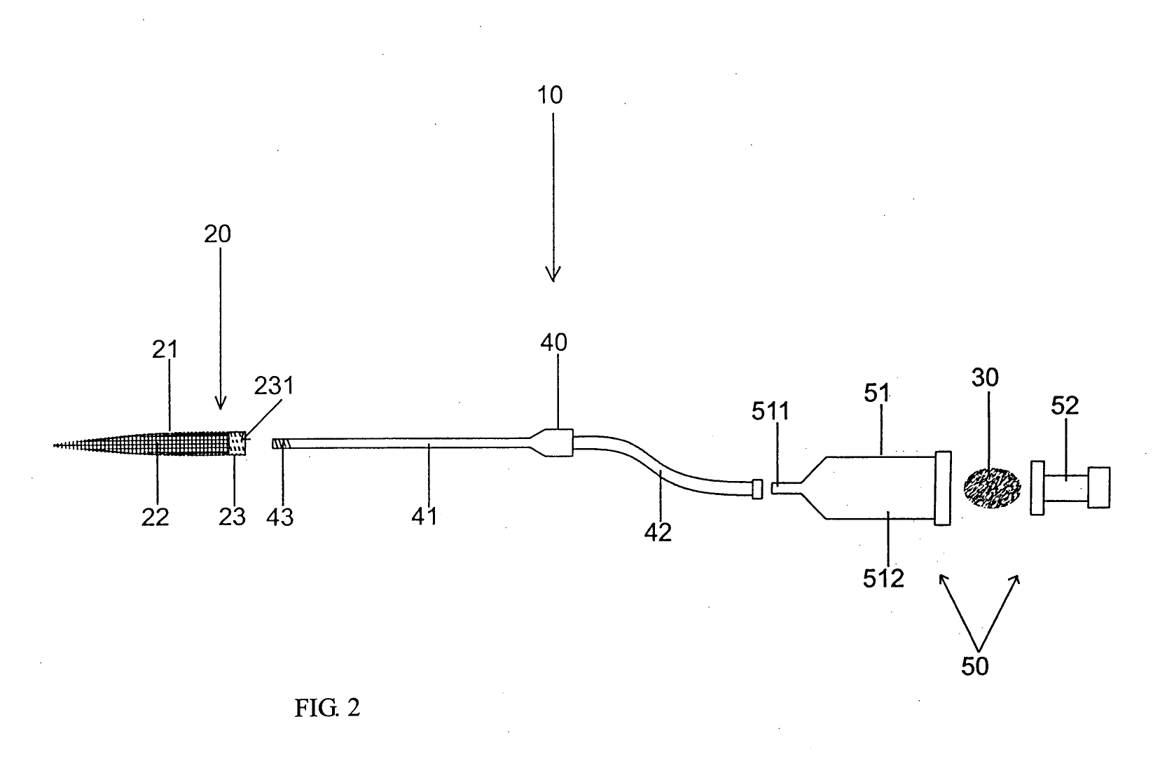

As shown in FIG. 2, the filling member 20 is integrally made of a flexible and breathable single-layer wall 21 by punching and pressing and is provided with a hollow receiving portion 22 and an inlet 23 in communication with the receiving portion 22. The inlet 23 is provided with an inner threaded portion 231.

The pasty medicinal material 30 is preferable capable pf hardening. Prior to being injected into the filling member 20, the pasty medicinal material 30 is held in a barrel 51 of the injecting cylinder 50. The material 30 is injected by a plunging motion of a plunger 52 into the filling member 20 via the connection tube 40. The connection tube 40 is provided at one end 41 with an outer threaded portion 43 and is fastened at the one end 41 with the inlet 23 of the filling member 20 in such a manner that the outer threaded portion 43 of the connection tube 40 is engaged with the inner threaded portion 231 of the inlet 23. The connection tube 40 is fastened at another end 42 with an outlet 511 of the barrel 51.

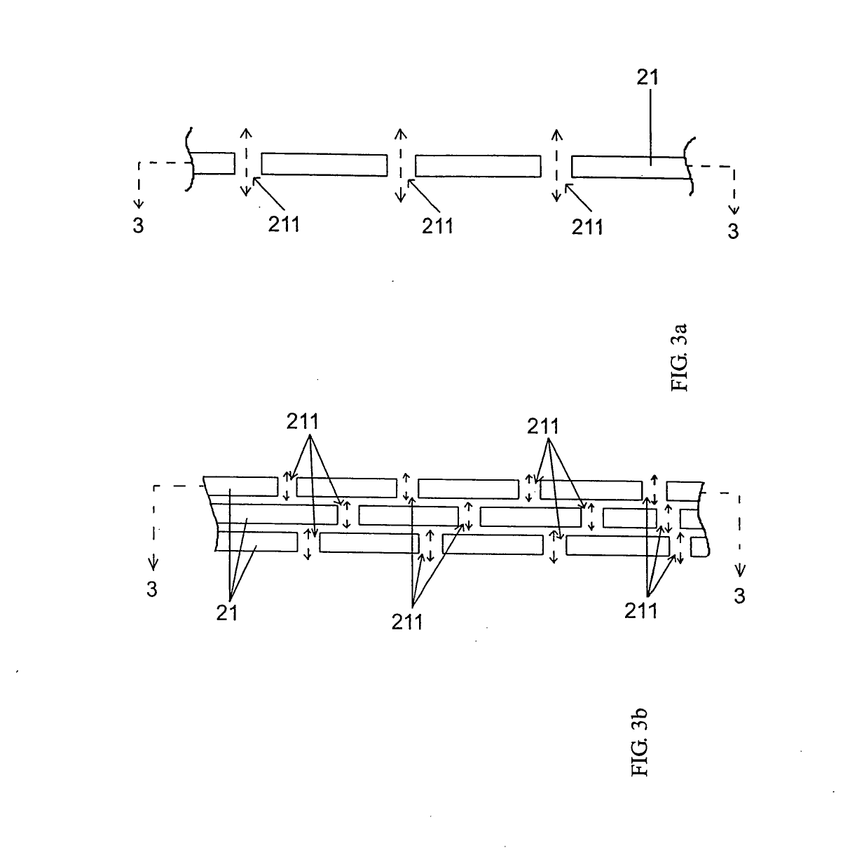

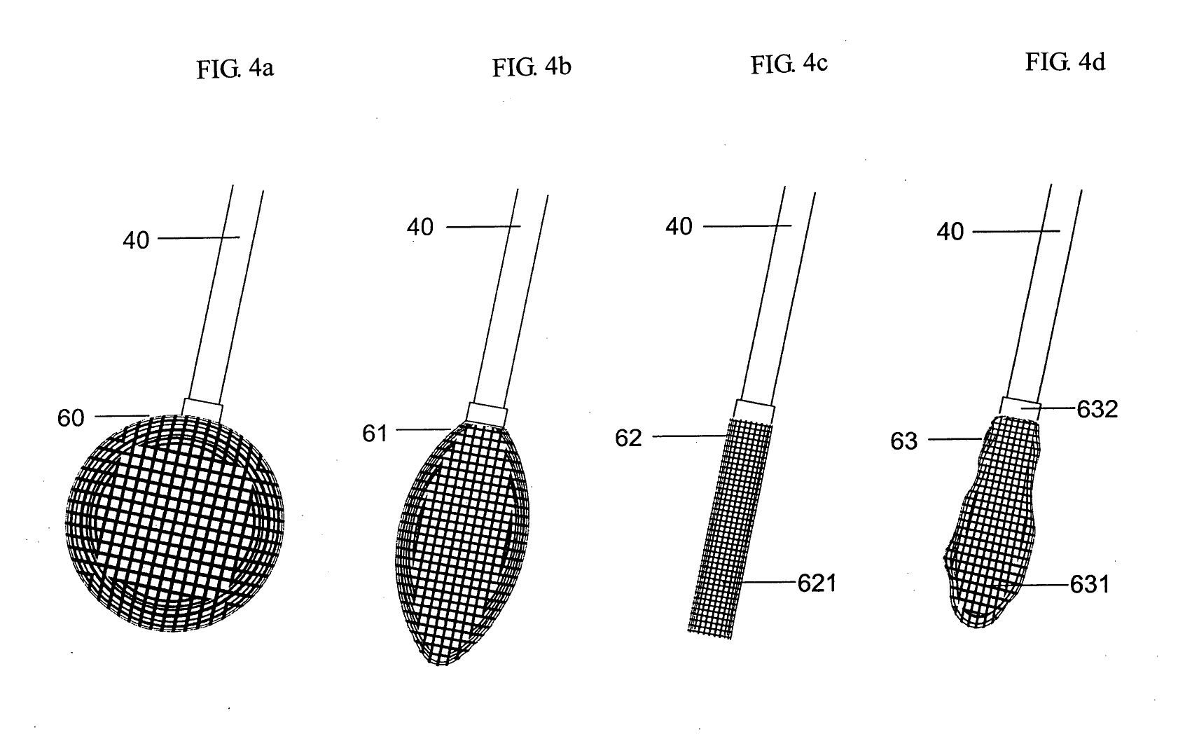

As shown in FIG. 3a, the wall 21 of the filling member 20 is formed of one layer. The wall 21 is provided with a plurality of meshes 211 pervious to gas or liquid and impermeable to solid. The filling member 20 is elastically compressible by virtue of the elasticity of the wall 21.

As shown in FIG. 3b, the wall 21 are multi-layer laminated wall, wherein the meshes 211 of two adjacent layers are not aligned. As a result, the passage of gas or liquid through the walls 21 is slowed down by the nonalignment of the meshes 211. The meshes 211 are impermeable to the solid constituent of the material 30, thereby resulting in the walls 21 being pushed by the solid constituent.

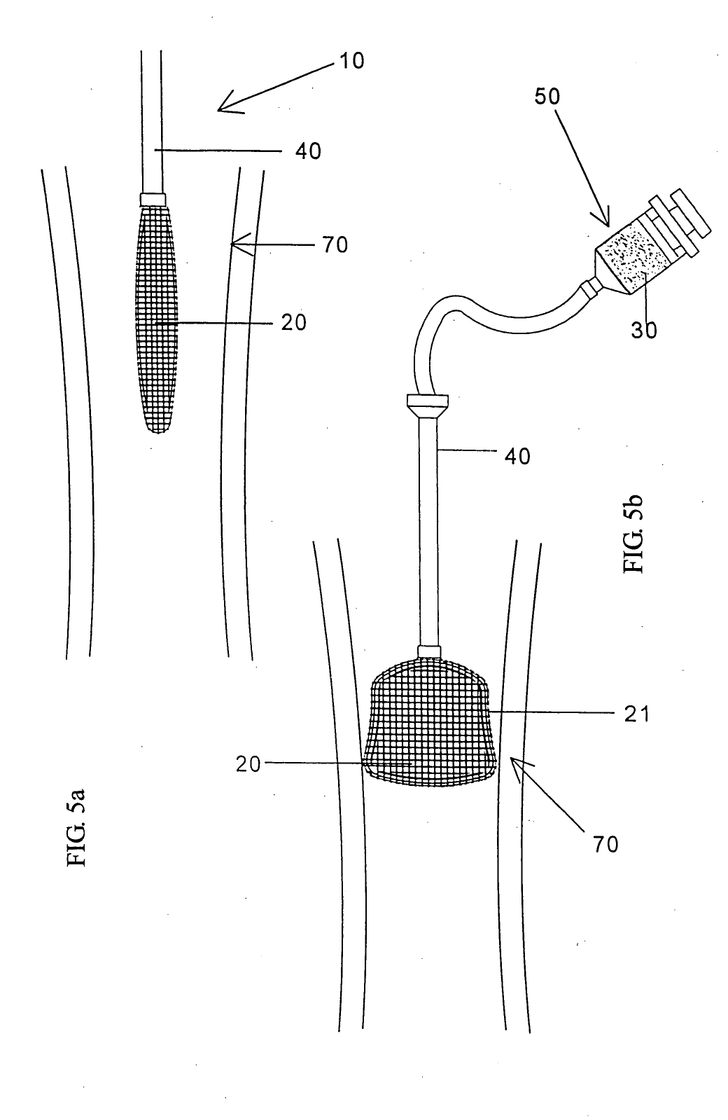

As illustrated in FIGS. 4a-4d, the filling member of the present invention takes various forms, such as spherical 60, oval 61, rod-shaped 62, and irregular-shaped 63. The rod-shaped member 62 is formed of a plurality of walls 621, which are so connected to form a rod shape. The irregularly-shaped member 63 is provided with a receiving portion 631 and an inlet 632.

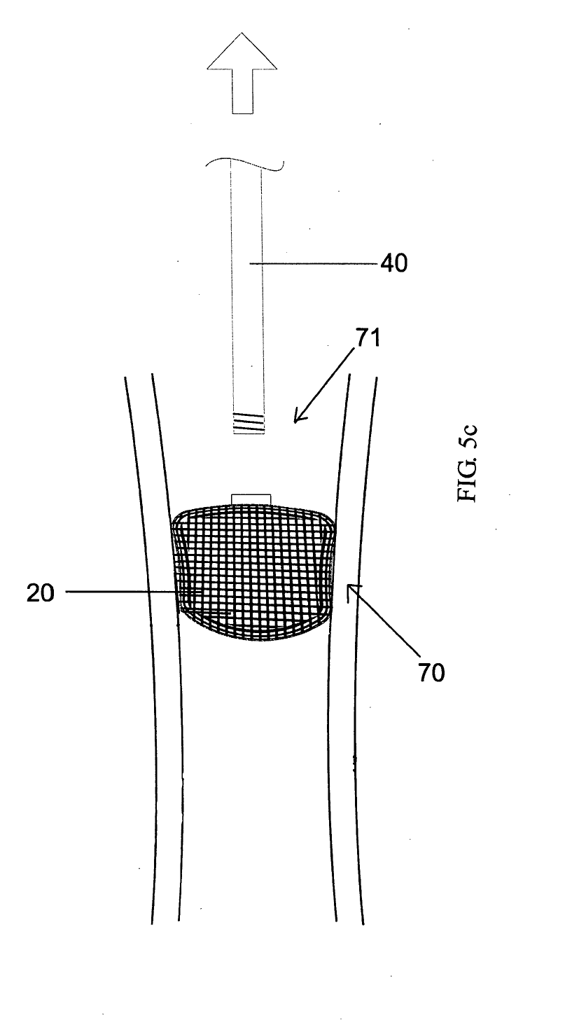

As illustrated in FIG. 5a, the filling member 20 is first compressed so as to enable it to be inserted into a bone cavity 70. Thereafter, the filling member 20 is filled with the pasty medicinal material 30, which causes the filling member 20 to regain its original form, as illustrated in FIG. 5b. As the pasty medicinal material 30 has hardened inside the filling member 20, the connection tube 40 is unfastened with the filling member 20, as illustrated in FIG. 5c. The bone cavity 70 is thus blocked by the filling member 20 so as to facilitate the planting of an artificial implant, such as an artificial hip joint, in an upper portion 71 of the bone cavity 70. The filling member 20 serves as a means to support the artificial hip joint.

The present invention described above is to be regarded in all respects as being illustrative and nonrestrictive.

Accordingly, the present invention may be embodied in other specific forms without deviating from the spirit thereof. The present invention is therefore to be limited only by the scopes of the following claims.

What is claimed is:

1. A method for implanting a medical filler into a bone comprising the following steps:
providing a flexible and breathable filling member formed of one or more meshed walls and provided with a hollow receiving portion and an inlet in communication with said receiving portion, said meshed walls being elastically compressible and a single-layer wall or a laminated multi-layer wall, and each layer of which is provided with more than 100 meshes per linear inch., so that said meshed walls are not air-tight;
forming an access opening on a bone so that a cancellous portion thereof is exposed;
directly inserting the filling member into the access opining so that the filling member is in the cancellous portion of the bone without using a tool to create a cavity larger than the access opening in the cancellous portion of the bone; and
injecting a pasty medicinal material into said receiving portion via said inlet of said filling member.

2. The method as defined in claim 1, wherein said meshed walls are of a single-layer wall construction.

3. The method as defined in claim 1, wherein said meshed walls are of a laminated multi-layer wall construction, and the meshes of each layer of the multi-layer walls are not aligned.

4. The method as defined in claim 1, wherein said filling member is integrally made of one meshed wall such that said filling member takes the form of a sac, bag, or ball.

5. The method as defined in claim 1, wherein said pasty medicinal material is a mixture of a liquid and a bone cement.

6. The method as defined in claim 5, wherein said bone cement is selected from the group consisting of gypsum, calcium sulfate, calcium phosphate, and hydroxy apatite.

7. The method as defined in claim 1 further comprising fastening detachably an injection tool with said inlet of said filling member, so that said pasty medicinal material is injected into said receiving portion via said inlet of said filling member by said injection tool.

8. The method as defined in claim 7, wherein said injecting tool comprises a connection tube and an injecting cylinder, said connection tube being provided at one end with an outer threaded portion such that said outer threaded portion of said connection tube is engaged with an inner threaded portion of said inlet of said filling member; said injecting cylinder comprising a barrel and a plunger, said barrel being fastened with another end of said connection tube, wherein said barrel is used to hold said pasty medicinal material which is to be injected into said receiving portion of said filling member via said connection tube by a plunging motion of said plunger.
